(19) United States
(12) Patent Application Publication
(10) Pub. No.: US 2010/0152488 A1
(43) Pub. Date: Jun. 17, 2010

(54) High Purity (-) Hydroxycitric Acid Metal Salt Derivatives and Method of Preparation of the Same

(75) Inventors: Gudapati Vendateswara Rao, Bangalore (IN); Alageri Chandrashekara Karunakara, Bangalore (IN); Mullakkapurath Narayanan Manoj, Bangalore (IN); Anil Kumar Kush, Bangalore (IN); Goukanapalli Chandrasekara Reddy, Bangalore (IN)

Correspondence Address: FISH & RICHARDSON P.C., P.O BOX 1022, Minneapolis, MN, 55440-1022, US

(73) Assignee: VITTAL MALLYA SCIENTIFIC RESEARCH FOUNDATION, Bangalore (IN)

(21) Appl. No.: 12/527,544

(22) PCT Filed: Aug. 28, 2007
(86) PCT No.: PCT/IN07/00371

§ 371 Date: Feb. 11, 2010

(30) Foreign Application Priority Data
Feb. 16, 2007 (IN) 325/CHE/2007

(51) Int. Cl. C07C 59/265; C07C 51/41

(52) U.S. Cl. 562/584

(57) ABSTRACT

The present invention relates to high purity (−) Hydroxycitric acid metal salt derivatives of more than 98% purity having formula II (a-e), lla; X, 2X═Na Mb; X, 2X═K Hc; X═K, Y═Ca Hd1 X═Na, Y═Ca He; X=½Mg, Y═Mg and the method of preparation of high purity (−) hydroxycitric acid metal salt derivatives from (−) hydroxycitric acid lactone having formula (1) which in turn is isolated in a simple and efficient manner from dried rinds of the fruits of Garcinia species. This invention employs the HPLC method and optical rotation in assessing the purity (−) hydroxycitric acid and corresponding lactone (1).

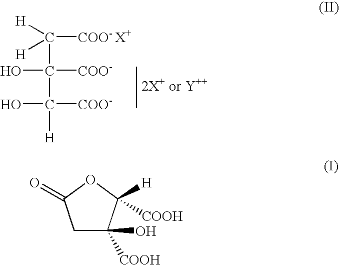

### FIELD OF INVENTION

The instant invention relates to novel high purity (−) Hydroxycitric acid metal salt derivatives of more than 98% purity and the process of preparation of the same.

### BACKGROUND OF THE INVENTION

(−)-Hydroxycitric acid (HCA) is a constituent present in the fruits of Garcinia cambogia, Garcinia indica and Garcinia atroviridis. Since HCA is labile and gets easily tactonised to form (−)-hydroxycitric acid lactone or 2S,3S-tetrahydro-3-hydroxy-5-oxo-2,3-furan dicarboxylic acid, it is generally isolated as its salts. (U.S. Pat. No. 6,147,228 dated Jul. 30, 1999; U.S. Pat. No. 6,160,172 dated Dec. 12, 2000 and US patent application publication 0137950 A1 dated Sep. 26, 2002 and references cited therein). In these inventions a soluble mono salt or double metal salts of group 1A and 11A of HCA having general formula (II) and lactone of formula (1) and its derivatives have been prepared.

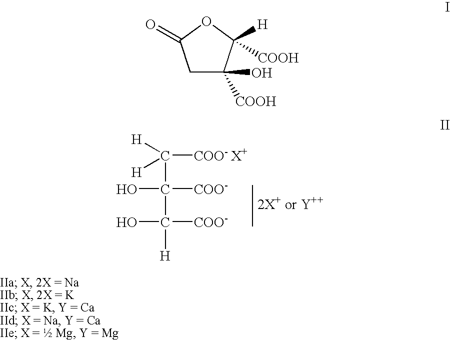

The steps involved are water extraction of Garcinia rind containing HCA and it's concentration, followed by purification using ion-exchange resins to get both free HCA and its lactone or by direct neutralization of aqueous extract with group IA metal hydroxides to get mono metal salt of (−) hydroxycitric acid. Then partial displacement of 1A group metal ions in above salt solutions by adding IIA metal chlorides to form soluble double metal salts of group 1A and IIA of (−) hydroxycitric acid.

The method for preparation of (−) hydroxycitric acid lactone by Y. S. Lewis and S. Neelakantan, Phytochemistry, Vol. 4, 1965, pages 619-625 & U.S. Pat. No. 6,147,228 involves extraction of dried Garcinia rind with hot water, concentration and conversion into tri sodium salt. This tri sodium salt was washed with aqueous alcohol several times and then converted into hydroxycitric acid by passing through cation exchange resin or by neutralization using hydrochloric acid. The solution thus obtained was evaporated to dryness and crystallized after drying for several hours.

Above methods suffer from batch to batch variation of (−) hydroxycitric acid content in raw material and hence it affects the composition of final products and their purity.

The main drawbacks of the existing methods are as follows:
1. It employs expensive and tedious ion exchange chromatography.
2. The salts made here are of less pure.
3. Possibility of metal halide contamination in final product.
4. There is no method described to establish the purity of any of the intermediates or (−) hydroxycitric acid and hence there is no way to add stoichiometric quantities of metal salts.
5. It employs laborious process of treating the syrup obtained after evaporation of aqueous extract several times using alcohols, followed by alkali treatment to obtain metal salts and then convert into (−) hydroxycitric acid lactone.
6. Neutralization of metal salts of (−) hydroxycitric acid by mineral acids/cation exchange resin before making lactone leads to generation of effluents.
7. Purity of lactone not established and physical constants are reported only partially.

### OBJECT OF THE INVENTION

Therefore the object of the present invention is to obviate the drawbacks of the prior art by providing high purity (−) Hydroxycitric acid metal salt derivatives and the process of preparation of the same.

### SUMMARY OF THE INVENTION

In order to achieve the said objectives the present invention provides a high purity (−) hydroxycitric acid metal salt of more than 98% purity derivatives having formula II (a-e).

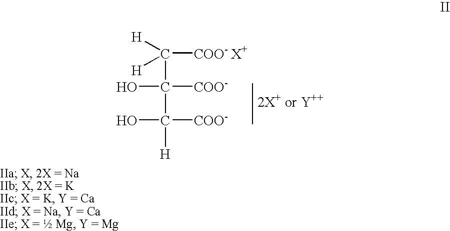

Further, the present invention provides a novel and simple method of preparation of high purity (−) hydroxyl citric acid metal salts having formula II (a-e) using (−) hydroxyl citric acid lactone of formula (1) isolated from Garcinia sp. comprising the steps of:
i) preparing (−) hydroxycitric acid concentrate from water extract of Garcinia and converting into its lactone having formula (1),
ii) hydrolyzing and neutralizing (−) hydroxycitric acid lactone using metal hydroxides or metal carbonates,
iii) precipitating and isolating said salts by adding alcohols to obtain pure salts of (−) hydroxycitric acid having a purity of above 98%.

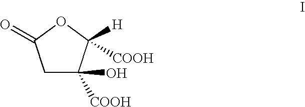

This invention employs the HPLC method and optical rotation in assessing the purity (−) hydroxycitric acid and corresponding lactone (1).

Optionally, the lactone of formula 1 is obtained from commercially available calcium, sodium or potassium salts (−) hydroxycitric acid or its aqueous solution comprising the steps of
neutralizing the, solution with HCl/H2SO4 and
evaporating the solution to dryness to get crude lactone.
purifying said lactone by crystallization using ethyl acetate/hexane mixture.

The present invention further provides a method of obtaining the pure (−) hydroxycitric acid lactone having formula (1), which is isolated in a simple and efficient manner from dried rinds of the fruits of Garcinia species comprising the steps of:
i. boiling the dried rinds of the fruits of Garcinia Cambogia and extracting into boiling water for 6-10 hours three times,
ii. evaporating the extracted mass to obtain a syrupy mass,
iii. adding acetone/methanol and their analogues as such or in different proportions to remove pectin and other insoluble matter.
iv. filtering the mass and filtrate is concentrated to obtain a viscous mass,
v. extracting the viscous mass repeatedly with organic solvents,
vi. evaporating the organic layer to obtain crude lactone,
vii. crystallizing said crude lactone from solvents to obtain pure crystals of (−) hydroxycitric acid lactone.

### DETAILED DESCRIPTION OF THE INVENTION

The following steps are involved in the present invention.

The dried rinds of the fruits of Garcinia Cambogia are cut into small pieces and extracted into boiling water for 6-10 hours three times. The combined extracts are evaporated to get a syrupy mass.

Sufficient quantity of acetone/methanol and their analogues like methyl ethyl ketone, isobutyl methyl ketone/ethyl alcohol/isopropyl alcohol/butyl alcohol as such or in different proportions added to remove pectin and other insoluble matter. After filtration, the filtrate is concentrated to get a viscous mass.

The viscous mass is repeatedly extracted with diethyl ether, di isopropyl ether, methyl tertiary butyl ether/butyl acetate, isopropyl acetate, ethyl acetate and on evaporation of organic layer yielded crude lactone.

The crude lactone upon crystallization from solvents like diethyl ether, ethyl acetate, acetonitrile with petroleum ether in different proportions gives pure crystals of (−) hydroxycitric acid lactone.

The purity of the product is confirmed by subjecting to HPLC. The optical rotation of the lactone is determined in ethyl acetate, water and methanol; the values are given in the table below.

| S. No | Solvent | Concentration | [α]²⁵_{D} |
|---|---|---|---|
| 1 | EtOAc | 1% w/v | +89.7⁰ |
| 2 | Water | 1% w/v | +112.6⁰ |
| 3 | Methanol | 1% w/v | +86.6⁰ |

The pure crystalline lactone has been dissolved in water and treated with stoichiometric quantities of alkali viz., sodium hydroxide, potassium hydroxide, calcium carbonate, potassium carbonate, magnesium carbonate and other IA and IIA group metal carbonates/hydroxides. These solutions when spray dried or precipitated with ethyl alcohol to get corresponding metal salts of (−) hydroxycitric acid.

The (−) hydroxycitric acid lactone has also been prepared from commercially available calcium/sodium/potassium salts and their solutions of hydroxycitric acid.

The present invention will now be explained with the help of examples however; the scope of the invention should not be limited to them.

### Example 1

### (2S,3S)-Tetrahydro-3-hydroxy-5-oxo-2,3-furandicarboxylic acid (I)

Dried Garcinia Cambogia fruit rinds were made into small pieces (200 g) and immersed in hot water (250 ml) for 10 hours. Water was decanted and the process was repeated for 3 times. The combined water extracts were concentrated to get a thick syrupy mass to which acetone was added. The precipitated mass was filtered off and washed with acetone. Acetone layer on evaporation gave a gummy mass, which was extracted with ethyl acetate. The ethyl acetate was charcolised, dried over anhydrous sodium sulphate, which on concentration gave crude (−) hydroxycitric acid lactone. This material was crystallized using ethyl acetate/n-hexane to yield material of high purity of compound I.

Alternatively compound I has been made from commercially available (−) hydroxycitric acid sodium salt or its aqueous solution by neutralizing with HCl/H2SO4 and evaporating the solution to dryness to get crude lactone. This tactone has been purified by crystallization using ethyl acetate/hexane mixture.

Yield: 25.0-30.0 gms

Melting point: 176-178° C.

[α]²⁵_{D}: +112.6° (C.=1.0 in water)

HPLC purity: >98%,

### Example 2

### Trisodium (2S,3S)-dihydroxy-1,2,3-propane tricarboxylate (IIa)

To an aqueous solution of I (1.9 g, 10 m mol, in 10 ml water) was added 1.10 g of sodium hydroxide in 5 ml water and heated to 60° C. for 2 hours. Further quantity of aqueous sodium hydroxide solution was added till the pH of the solution is neutral. The resultant solution was filtered and the material was precipitated using ethyl alcohol. The product IIa was finally dried under vacuum

Yield: 2.5 g

HCA content: 73.5 to 74.5%

Sodium content: 24.5 to 25.2%

### Example 3

### Tripotassium (2S,3S)-dihydroxy 1,2,3-propane tricarboxylate (IIb)

To an aqueous solution of I (1.9g, 10 mmol in 10 ml water) was added 1.60 g of potassium hydroxide in 5 ml water and heated at 60° C. for 2 hours. Then it was cooled and adjusted its pH to neutral with aq.potassium hydroxide solution. The resultant solution was filtered and the material was precipitated using ethyl alcohol. The product IIb was finally dried under vacuum.

Yield: 2.7 g

HCA content: 62.5 to 63.1%

Potassium content: 35.5 to 36.4%

### Example 4

### Monopotassium, calcium (2S,3S)-dihydroxy 1,2,3-propane tricarboxylate (IIc)

To an aqueous solution of I (1.9 g, 10 mmol in 10 ml water) was added 1.0 g of calcium carbonate in 5 ml water stirred at room temperature for 2 hours and then heated to 60° C. for 1 hour. Cooled to room temperature and added 0.55 g of potassium hydroxide in 5 ml water. Then the pH of the solution adjusted to neutral with potassium hydroxide. The resultant solution was filtered and the material was precipitated using ethyl alcohol. The product (IIc) was finally dried under vacuum.

Yield: 2.6 g

HCA content: 70.9 to 71.5%

Potassium content: 16.32 to 16.8%

Calcium content: 11.9 to 12.3%

### Example 5

### Monosodium, calcium (2S,3S)-dihydroxy 1,2,3-propane tricarboxylate (IId)

To an aqueous solution of I (1.9 g, 10 mmol in 10 ml water) was added 1.0 g of calcium carbonate in 5 ml water, stirred at room temperature for 2 hours and then, heated to 60° C. for 1 hour. Cooled to room temperature and then added 0.40 g of sodium hydroxide in 5 ml water. Then the pH of the solution was adjusted to neutral with sodium hydroxide. The resultant solution was filtered and the material was precipitated using ethyl alcohol. The product (IId) was finally dried under vacuum.

Yield: 2.5 g

HCA content: 75.1 to 75.9%

Sodium content: 8.9 to 9.1%

Calcium content: 14.3 to 14.6%

### Example 6

### Magnesium (2S,3S)-dihydroxy 1,2,3-propane tricarboxylate (IIe)

To an aqueous solution of I (1.9 g, 10 mmol in 10 ml water) was added 1.0 g of magnesium carbonate in 5 ml water, stirred at room temperature for 2 hours and then heated to 60° C. for 1 hour. Added magnesium carbonate to the solution till pH became neutral. The resultant solution was filtered and the material was precipitated using ethyl alcohol. The product (IIe) was finally dried under vacuum.

Yield: 2.2 g

HCA content: 83.6 to 84.8%

Magnesium content: 14.5 to 15.0%

Advantages of New Process

In the context of stringent regulations being enforced on the nutraceuticals with respect to quality and reproducibility, it is relevant to investigate new methodologies.

The present invention is an efficient and simple alternative route for making (−) hydroxycitric acid derivatives in a highly pure state avoiding ion exchange chromatography.

This method provides a simple, economical and efficient method of obtaining crystals of (−) hydroxycitric acid lactone with HPLC purity of more than 98% (area normalization method). Further this pure lactone is being used to make all derivatives.

This process does not involve extra steps of basification/neutralization, thus reducing the effluent burden.

Since pure salts are being made, it would be convenient to mix them in desired proportions to get metal content of choice.

The process also provides HPLC method combined with optical rotation measurements to check the ratios of pure acid vs lactone in a given preparation.

Most significantly the present invention delivers a chemically definable form of (−) Hydroxycitric acid suitable for pharmaceutical formulations.

1. High purity (−) Hydroxycitric acid metal salt derivatives of more than 98% purity having formula II (a-e);

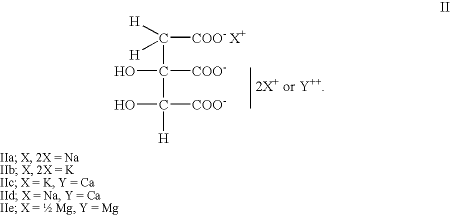

2. A method of preparing high purity (−) hydroxy citric acid metal salts having formula II (a-e) using (−) hydroxy citric acid lactone having formula (1) isolated from Garcinia sp., comprising the steps of:
a. preparing a (−) hydroxycitric acid lactone from a water extract of Garcinia and converting (−) hydroxycitric acid into its hydroxycitric acid lactone having formula (1),
b. hydrolyzing and neutralizing the (−) hydroxycitric acid lactone having formula (1) using metal hydroxides or metal carbonates,
c. precipitating and isolating said the hydroxycitric acid metal salts by adding alcohols to obtain pure salts of (−) hydroxycitric acid having a purity of above 98%.

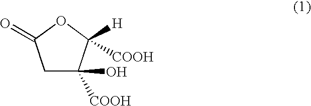

3. The method of claim 2, wherein the precipitation and isolation in step c is done with ethyl alcohol.

4. The method of claim 2, further comprising obtaining pure lactone crystals comprising the steps of:
a. boiling dried rinds of fruits of Garcinia Cambogia and extracting into boiling water for 6-10 hours three times to obtain a combined extract;
b. evaporating the combined extract to obtain a syrupy mass;
c. adding acetone/methanol and their analogues as such or in different proportions to remove pectin and other insoluble matter;
d. filtering the syrupy mass and concentrating the filtrate to obtain a viscous mass;
e. extracting the viscous mass repeatedly with organic solvents;
f. evaporating the organic solvents to obtain crude lactone; and
g. crystallizing the crude lactone from solvents to obtain the pure crystals of (−) hydroxycitric acid lactone.

5. The method of claim 4, wherein the analogues of acetone/methanol are selected from the group consisting of methyl ethyl keton, and isobutyl methyl ketone/ethyl alcohol/isopropyl alcohol/butyl alcohol.

6. The method of claim 4, wherein the organic solvents are selected from the group consisting of diethyl ether, di-isopropyl ether, methyl tertiary butyl ether/butyl acetane, isopropyl acetate, and ethyl acetate.

7. The method of claim 4, wherein the solvents for crystallization are selected from the group consisting of diethyl ether, ethyl acetate, hexane, acetonitrile, diethyl ether, di-isopropyl ether and acetonitrile.

8. The method of claim 7, wherein the solvents for crystallization are hexane and ethyl acetate.

9. A method of preparing the high purity (−) hydroxyl citric acid metal salt of claim 1, wherein the salts are soluble single, double and/or poly metal salts.

10. A method of preparing high purity (−) hydroxy citric acid metal salts having formula II (a-e), wherein the (−) hydroxycitric acid lactone having formula 1 is obtained from commercially available calcium, sodium, or potassium salts of (−) hydroxycitric acid or its aqueous solution, comprising the steps of:
a. obtaining a (−) hydroxycitric acid lactone, comprising:
i. neutralizing the solution with HCl/H₂SO₄;
ii. evaporating the solution to dryness to get crude lactone; and
iii. purifying the crude lactone by crystallization using an ethyl acetate/hexane mixture,
b. hydrolyzing and neutralizing the (−) hydroxycitric acid lactone having formula (1) using metal hydroxides or metal carbonates, and
c. precipitating and isolating the hydroxycitric acid metal salts by adding alcohols to obtain pure salts of (−) hydroxycitric acid having a purity of above 98%.

11. The method of claim 10, wherein the metals of the metal hydroxides or metal carbonates are one or more from the group consisting of Sodium, Potassium, Calcium, Magnesium, Zinc, Manganese, Selenium, Germanium, and Cobalt.

12. (canceled)

13. (canceled)
